(19) United States
(12) Patent Application Publication
(10) Pub. No.: US 2023/0141419 A1
(43) Pub. Date: May 11, 2023

(54) MACHINE LEARNING-BASED ACTIVITY AND PROGRAM RECOMMENDER

(71) Applicant: League, Inc., Toronto (CA)

(72) Inventors: Kyle Thompson, Toronto (CA); Kerry Weinberg, Waltham, MA (US); Yasmeen Almog, New York, NY (US)

(21) Appl. No.: 17/981,552

(22) Filed: Nov. 7, 2022

Related U.S. Application Data
(60) Provisional application No. 63/277,135, filed on Nov. 8, 2021.

(51) Int. Cl. G16H 20/30; G16H 20/60

(52) U.S. Cl. CPC G16H 20/30; G16H 20/60

(57) ABSTRACT

Probability models of user behaviour are generated by tracking and recording user progress through health programs consisting of individual activities. Historical user engagement with a program or activities is aggregated into probability models that estimate future user behaviours and engagement with different activities. Using probability models, a next best engagement engine identifies and suggests health programs that have been determined to maximize the aggregate overall expected value to the user of being presented with an activity sequence. To model the probability of a user completing different activities in the future given past user behaviours, machine-learning techniques that train on input data sets observed over time are utilized. When combined with functions that are generated to model the value to be derived from completing different activities or combinations of activities, together with user demographic data, such probability models can be used to compute optimal activity sequences for individual users.

### CROSS-REFERENCE TO RELATED APPLICATIONS

This application claims priority to U.S. Application Ser. No. 63/277,135, filed on Nov. 8, 2021, and entitled MACHINE LEARNING-BASED ACTIVITY AND PROGRAM RECOMMENDER, the entire contents of which are hereby incorporated by reference.

### TECHNICAL FIELD

The disclosure relates generally to digital content delivery, and, in particular, to the use of machine-learning techniques for dynamically generating and presenting personalized health and wellness content to a user of a device via a user application.

### BACKGROUND

There is much attention and interest currently on the well-being and general state of people's health. Employers, benefit providers, and targeted health solutions providers (e.g., in the fields of mental health, sleep, diabetes, virtual care, etc.) are offering their members and subscribers programs to address demand for improved health and wellbeing, such as physical fitness, management of medical conditions, general lifestyle, and other areas of life. However, user engagement in such programs remains low and faces obstacles. Increased user engagement in health-related service offerings would positively contribute to the overall wellbeing and state of people's health.

### BRIEF DESCRIPTION OF THE DRAWINGS

Reference will be made herein to the accompanying drawings, in which:

FIG. 1 illustrates a directed acyclic graph that may be used to model dynamic health programs in accordance with the disclosure.

FIG. 2 illustrates the use of user activity and orchestration windows to present dynamically generated health programs to users in accordance with the disclosure.

FIG. 3 illustrates an example architecture for a content recommendation system in accordance with the disclosure.

FIG. 4 illustrates an example implementation of the content recommendation system shown in FIG. 3.

FIG. 5 illustrates a method of delivering a health program to a user in accordance with the disclosure.

For clarity and ease of description, like reference numerals will be used in the drawings to describe the same or like parts.

### DETAILED DESCRIPTION

The description which follows, and the embodiments disclosed, are provided by way of illustration of example embodiments of the disclosed invention. These examples are provided for the purposes of explanation and are not intended to provide an exhaustive description of the disclosed embodiments and principles of the invention.

Current approaches to the delivery of health-related programs or campaigns have low user engagement in part due to a lack of content personalization for individual users and challenges associated with offering customized programs to a wide audience. For example, while a single program may be customized or designed specifically for a particular individual, it may be the case that without modification the program will not be suitable for or of interest to other individuals that have different characteristics and personalized health goals. Conversely, an individual program that is targeted or designed to appeal to a broader audience may not necessarily advance individual health objectives as effectively as customized programs would or even at all. At the same time, designing customized health programs for each and every individual that is part of a wider audience may be time consuming and not practically feasible.

An additional complexity is that customization or personalization of health programs may in some cases also involve the provision of periodic updates or program adjustments. For example, a user's personal characteristics or circumstances can change over time. Program adjustments may also be called for if, for example, the user is not engaging with the program as anticipated or because the user's goals have shifted over time. In some instances, the program's original design may have been a poor fit for the user. Programs that prescribe only a linear sequence of activities to be performed by the participant without the possibility of variation or adjustment based on feedback received from the user are unlikely to respond effectively to any of the above noted scenarios. Consequently, user engagement with programs of this general structure may be low and/or impacted by negative user experience.

The present disclosure is directed to providing systems and methods that enable the dynamic generation of recommendations to individuals of available health programs, activities, services, and other health-related content. The recommendations provided to the individual can form a personalized health program or plan that is intended by the creator(s) of the content to lead to an overall expected improvement of the individual's health and wellbeing, manage medical conditions, diseases, or ailments, and/or permit the individual to develop physical and mental habits that would lead to an expected improvement of overall health and wellbeing.

Dynamically generated programs can be personalized for each individual user and adjusted or modified over time through the use of branching logic and activity parameterization for the user as described herein. As compared to a static, linear sequence of activities, dynamic programs having this general structure may usefully take into account a user's demographic and/or historical engagement data when selecting particular activity sequences for the user from among different permutations. By taking these factors and data into account, dynamic programs can be generated for each different user individually through customized selections of activities from among a common activity pool in order to maximize the computed or estimated health benefit or engagement that each user can be expected to derive from participating in a program or set of programs. By combining activity parameterization with user data, dynamically generated programs can be both personalized for individuals and appealing to a broad audience of participants at the same time.

The disclosed systems and methods may be useful, for example, in the context of a group plan or an employer-provided health benefit regime in which various insurer-provided benefits, health programs, products, and services (collectively “health benefits”) are available to a covered employee or group plan member. The disclosed systems and methods can be used in this example context to more effectively identify and/or recommend health-related content for the covered employee or group plan member that, when followed, may lead to positive or more positive health outcomes for the individual. Alternatively, in different contexts, the disclosed systems and methods may be effectively used to provide health-related content to subscribers of targeted health solutions providers in different fields of fitness, health, wellness, and condition management as examples.

As described herein, health programs can in some cases be designed to include or incorporate sequences of inter-related health activities that are delivered to the user in a dynamically structured manner to achieve a specific user goal or targeting a specific area of interest for the user, such as general health, mental health, physical health, financial health, and/or family health. Examples of specific goals can include weight loss, increasing flexibility and strength, diets or nutritional programs, learning new skills or sports, day to day management of medical conditions, and so on. The target goal of the user may be achieved or at least furthered by following the health program so as to learn and adopt new health-related behaviour or behaviours over time.

In some embodiments, a health program can include a sequence of activities that is presented to the user according to a defined timing or schedule, in which the particular sequence of activities that is presented to the user has been determined to represent the optimal sequence for the user as computed using a suitable metric, such as a cost or value function. The set of all possible sequences that form a dynamic program, from which the optimal sequence for the user may be computed, can include both “branching” sequences (i.e., the trajectory of the activity sequence may diverge conditionally or contingently based on observed input data or other factors) and “parameterized” sequences (i.e., different versions of a particular activity can be recommended to the user).

Multiple different health programs can be combined or organized into an overall health plan for the user comprising two or more health programs in which the user enrolls or participates (either sequentially one after the other or simultaneously) to pursue different health objectives. In general, there is no limit on the number of different programs that may be offered to the user as part of an overall health plan. In some cases, isolated activities that are not part of any specific program can also be recommended to the user and/or incorporated into an overall health plan.

A user's participation in or progress through a given health program or individual activities can be tracked and recorded. Such historical user engagement data with a program or activities can be aggregated into probability models that represent an estimate or prediction of future user behaviours and engagement with programs and/or activities. Once generated, probability models of user behaviour can subsequently be used to recommend activities or programs to the user by computing measures of expected value or engagement for the user of being presented with different sequences of activities to complete. Tracking of user participation or program can be performed either by way of self-reporting, for example, or alternatively with the use of telemetric devices, such as a smart watches, health or activity trackers, biometric sensors or monitors, and other wearable devices that capture biometric data (e.g. heart rate, sleep habits, blood pressure, etc.).

In accordance with the disclosure, a next best engagement or recommendation engine can be utilized in different embodiments to identify and suggest health programs that have been determined or computed to maximize the overall value, aggregated over time, which the user can be expected to derive from completing the different activities or programs that have been presented or recommended. As described in more detail herein, such recommendations by the next best engagement engine can be generated based on machine-learning techniques that train on input data sets that are observed over time, including historical user engagement data, in order to model the probability of a user completing different activities in the future given the user's static demographic data and past user behaviours. When combined with functions that are generated to model the value to be derived from completing different activities or combinations of activities, the probability models can be used by the next best engagement or recommendation engine to compute optimal activity sequences for individual users.

Use of a recommendation engine as disclosed herein improves the functioning of computer systems by enabling the generation of customized program and/or activity recommendations for users from common program or activity definitions. Use of customized recommendations advantageously can be expected to progress the users toward respective health, fitness, lifestyle, and wellness goals. Because a recommendation engine as disclosed herein trains on historical user data that is observed over time concerning which activities users do and do not complete, as more input data is observed, the system is also likely to generate increasingly beneficial recommendations for users based on increasingly accurate probability models and value functions. A further improvement provided by a recommendation engine as disclosed herein is that the probability model and/or engagement value functions used to make activity and program recommendations are customizable to meet different use cases, contexts, or applications both in how value to the user can be defined and through observation of potentially different input data sets.

### Directed Acyclic Graphs

In accordance with the disclosed embodiments, dynamic health programs can be modelled mathematically as a directed acyclic graph (DAG) denoted herein throughout by the symbol G. Each DAG used to represent a dynamic health program can comprise a plurality of two or more nodes (or vertices) V, each of which may be connected to at least one other adjacent downstream node by way of a corresponding directional edge E. The set of all nodes V and directional edges E define a plurality of different “paths” or “routes” through the DAG, commencing at a start node and terminating at one or more end nodes, by visiting adjacent nodes in sequence along connecting edges in their direction of traversal.

Each node V in a DAG used to model a dynamic health program may define or be associated with one or more different activities that potentially may be presented to the user as part of the program at a given point in the sequence. A given node V may be only associated with a single activity, in which case every path through the DAG passing through that node may correspond to a health program that incorporates the single available activity. In other cases, where a given node V is associated with two or more potential activities or versions of an activity, i.e., “parameterized” activities, each path through the DAG that visits the node may involve a selection of one (or in some cases more than one) activity from the set of potential activities associated with that node. For convenience, as used herein throughout, each such path may be considered as separate or distinct from each other path through the DAG that visits the identical set of nodes V (but are associated with different activity sequences). Examples of activities that may be associated at a node V can include different physical activities (e.g., exercises to complete), dietary activities (e.g., food restrictions), mental activities (e.g., meditations), and the like.

Each edge E in a DAG used to model a dynamic health program may be associated with logic, such as a condition or criteria, which defines a relationship between activities associated with adjacent downstream nodes. In some cases, the logic may define parallel paths through the DAG, i.e., “branching” paths, which are specific to different user demographics (e.g., gender, age) or health conditions (e.g., diabetes, heart disease, history of smoking). Branching based on user demographic data allows a particular health program to be configurable for a plurality of different user characteristics or traits. In other cases, the logic may define parallel paths through the DAG that are based on historical user engagement data (e.g., completion status) or on data or metrics collected from a telemetric device (e.g., completion time, energy output, speed). This can allow for adjustment or modification to a particular health on the fly based on how a user has been responding to recommendations. For example, as described herein, observations of past historical user data can be used to model future user behaviour and to predict activity sequences that will optimize value for the user.

Each individual health program can be represented by a corresponding DAG denoted Gⱼ=(Vⱼ, Eⱼ). In this notation, graph Gⱼ associated with a given health program can be defined by a corresponding set of vertices Vⱼ and associated edges Eⱼ, in any arbitrary number and relation depending on the structure of the health program. Further, each graph Gⱼ corresponding to a health program can be associated with a function gⱼ: Vⱼ→P({aⱼ}^{m}ᵢ₌₁), where P({aⱼ}^{m}ᵢ₌₁) represents the power set of all activities that can be selected at each node Eⱼ in graph Gⱼ. With this notation, each respective path or route through graph Gⱼ represents a corresponding ordered sequence of activities that can be selected from among the set of all ordered activities gⱼ across all nodes Vⱼ and associated edges E.

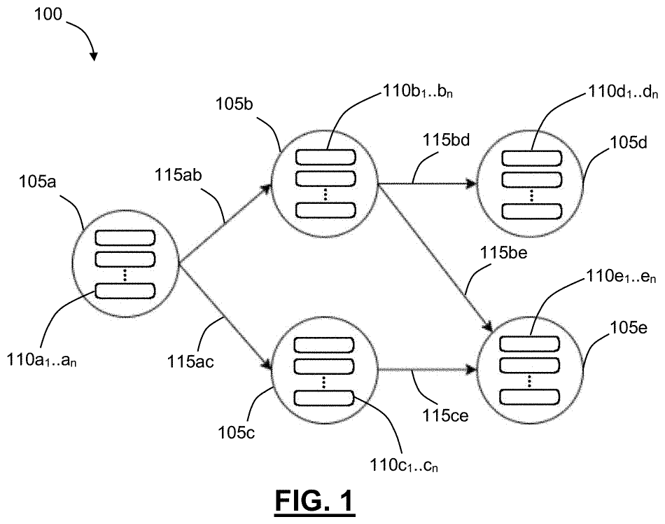

FIG. 1 shows an example of a directed acyclic graph 100 that can be used to model a dynamic health program as described herein. As illustrated, DAG 100 includes five individual nodes comprising a start node 105a from which all paths through DAG 100 will commence, intermediate nodes 105b and 105c, and end nodes 105d and 105e at which all paths through DAG 100 will eventually terminate. The number of nodes shown in DAG 100 is for purposes of illustration only and is not generally limited.

Each node 105 in DAG 100 is associated with a corresponding set of activities 110. For example, start node 105a is associated with activities 110a₁ . . . aₙ, which can be any arbitrary number depending on the health program being modeled by DAG 100. Likewise, intermediate nodes 105b and 105c are associated respectively with activities 110bᵢ . . . bₙ and 110cᵢ . . . cₙ, and end nodes 105d and 105e in DAG 100 are associated respectively with activities 110dᵢ . . . dₙ and 110eᵢ . . . eₙ, each of which may also be of any arbitrary and/or different size.

Start node 105a is connected to intermediate node 105b by way of edge 115ab and to intermediate node 105c by way of edge 115ac. Intermediate edge 105b is connected to both end nodes 105d and 105e by way of edges 115bd and 115be, respectively. Intermediate node 105c is connected only to end node 105e by way of edge 115ce. Thus, in this example, both intermediate nodes 105b and 105c connect to end node 105e by way of corresponding edges.

The number of unique or different paths through DAG 100 is defined by the arrangement of edges between nodes and the power set of activities available at each node. In the example shown, there are three unique traversals of nodes through DAG 100 that can be visited in order from start node 105a to one of the end node 105d,105e. These are T1={105a, 105b, 105d}, T2={105a,105b,105e}, and T3={105a,105c,105e}. Each respective traversal of nodes also itself provides multiple different paths through DAG 100 corresponding to different combinations of activities that can be selected at each given node. For example, traversal T1 can provide a total of aₙ×bₙ×dₙ different paths through DAG 100, traversal T2 can provide a total of aₙ×bₙ×eₙ different paths, and so on over all possible traversals of nodes along corresponding associated edges.

With the example structure given, DAG 100 can model a health program that will comprise a sequence of three individual activities, where the first activity is selected from among activities 110aᵢ . . . aₙ. In this example, on account of how the health program being modeled by DAG 100 has been defined, there will then be a choice to present the user with a second activity either from among activities 110bᵢ . . . bₙ or, alternatively, from activities 110cᵢ . . . cₙ, to allow for program personalization or customization based on different factors such as user demographics and/or historical user behaviour. If the second activity for the user was selected from activities 110bᵢ . . . bₙ, the third activity will then likewise be chosen from among activities 110dᵢ . . . dₙ or, alternatively, from activities 110eᵢ . . . eₙ. On the other hand, if the second activity was selected from activities 110cᵢ . . . cₙ, then the third activity will be selected from activities 110e₁ . . . eₙ.

As one example, the health program modelled may DAG 100 may have been defined so that the second activity presented to the user will depend on user demographic data, such as whether the user has a particular medical condition like diabetes. In that case, users with diabetes could branch to node 105b and be presented with one of activities 110bᵢ . . . bₙ, while users without diabetes could branch to node 105c and be presented with one of activities 110c₁ . . . cₙ. Other demographic data or factors such as age, could also in some cases form the basis of branching logic in DAG 100 generally without limitation.

As a second example, the user could branch to either node 105b or 105c depending instead on data related to the previous activity presented at node 105a, such as how quickly or easily the user completed the activity or whether it was completed at all. In that case, users who completed the activity at node 105a quickly or easily could branch to node 105b and be presented with a choice of relatively more difficult activities 110bᵢ . . . bₙ, while users who did not complete the activity quickly or easily or at all could branch to node 105c and be presented with relatively less difficult activities 110cᵢ . . . cₙ. Being able to customize the difficulty of the next activity to be completed based on information about the previous activity generally increases the possibility of recommending a next activity that the user will actually complete.

The example health program modelled by DAG 100 in FIG. 1 is dynamic in that each different user can in general be presented with different combinations of first, second, and third activities that are customized or personalized for the user, for example, in order to optimize for user engagement or value as described herein., i.e., to select the particular combination of activities that the user is predicted to complete and/or derive the most benefit from most often. Additionally, the health program modelled by DAG 100 is dynamic in that activity sequences (corresponding to paths through the DAG 100) do not necessarily have to be determined all at once and may instead be determined and/or modified incrementally in real time, i.e., while the user is progressing through the sequence. For example, as noted, the next activity in a sequence may be left undefined until and depending on how the previous activity by the user has been completed.

### Orchestration and User Activity Windows

Within a dynamic health program, the determination of new activities to present to the user can be controlled or scheduled with the use of one or more sliding windows that define limited time horizons into the future. For example, one or both of a user activity window and an orchestration window can be employed. A user activity window can define a partial sequence of activities of some arbitrary length that have been determined and are currently being presented to the user. In some cases, activities that have been presented within the user activity window will not be modified or replaced with newly determined activities, i.e., the partial sequence of activities appearing in the user activity window is static.

In addition to a user activity window, an orchestration window may be used to define a second partial sequence of activities, also of arbitrary length, which follows on from the partial sequence of activities appearing in the user activity window and includes activities that have been determined for the user but not yet presented. Activities that initially populate the orchestration window may be transferred into the user activity window over time as activities in the user activity window are completed or dismissed by the user. Thus, the user activity and orchestration windows may jointly define a time horizon into the future in which activities for the user have been determined, of which a subset of those activities are presented to the user at any given time in the user activity window. Activities in the orchestration window, unlike the user activity window, may in some cases be modified or changed at different points in time after having been initially determined, i.e., the partial sequence of activities appearing in the orchestration window is only quasi-static.

In general, the user activity window and orchestration window may each advance (“slide”) forward in time as a user progresses through a health program and completes or dismisses activities. The user activity window may therefore in some cases always start in the present time, denoted herein throughout as day k, and project forward in time a fixed number of increments, such as days or activities, depending on its chosen length. For convenience and ease of description, it is assumed that one activity is presented to the user per day, in which the length of the user activity and orchestration windows may each equivalently be defined in terms of days or activities, although this need not be the case. The orchestration window may start when the user activity window ends and slide forward in time in unison. However, the length of the orchestration window may be variable depending on the frequency of activity re-population. In some cases, the orchestration window may be re-populated at a lower rate (corresponding to a longer time internal) than the rate at which activities are transferred over time into the user activity window, which may cause the length of the orchestration window, i.e., the number of days or activities that it covers, to gradually decrease until such time as the orchestration window is re-populated with new activities. Alternatively, a new activity may be determined and inserted into the orchestration window for each activity that is removed and transferred over into the user activity window, in which case the orchestration window may also have a fixed length or size as defined in terms of either days or activities.

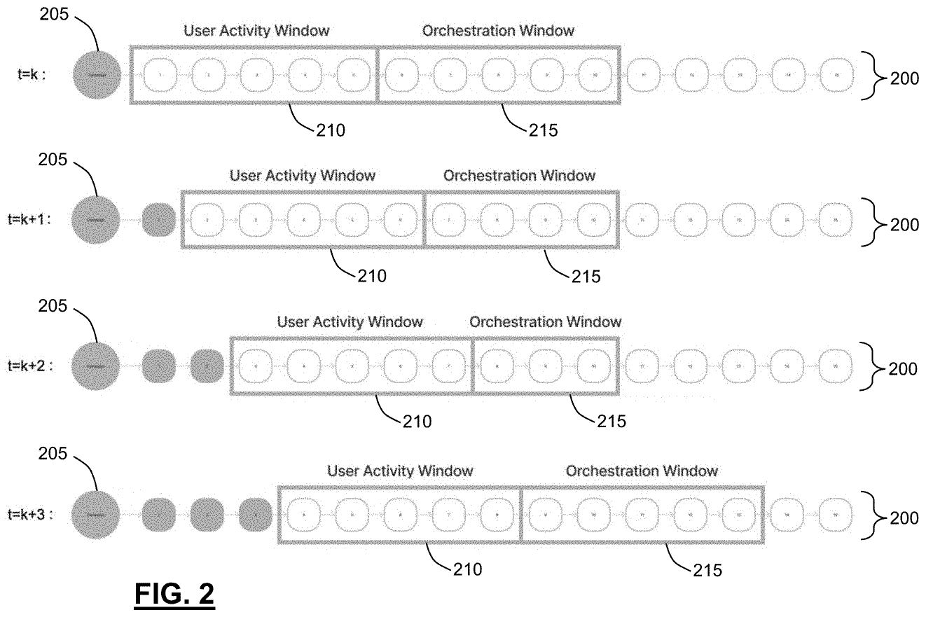

FIG. 2 shows an example activity sequence 200 that has been partially determined with the use of user activity and orchestration windows as described herein. Activity sequence 200 belongs to an example dynamic health program 205 that includes 15 different activities, starting on day k. The individual activities that form activity sequence 200 will be determined so as to optimize value for the user as described herein taking into account user demographic and historical user behaviour data.

In this example, ten activities are initially determined for the user, at time t=k, of which activities 1-5 are presented in user activity window 210 and activities 6-10 (although known) are hidden from the user for the time being within orchestration window 215. Activities 11-15 in sequence 200 are initially unknown at time t=k and have not yet been determined. At least initially, both user activity window 210 and orchestration window 215 each span a partial sequence of five activities. Activities 1-5 in user activity window 210 are static and will not be modified once determined. Activities 6-10 in orchestration window 215 are quasi-static and, in some cases, as described herein, may be modified or re-computed in real time following their initial determination at time t=k.

In this example, after the user has either completed or dismissed the first activity, at time t=k+1, user activity window 210 advances to the next activity in sequence 200. Likewise, at time t=k+2, after the user has completed or dismissed another activity, user activity window 210 again advances to the next activity in the sequence 200. At each step, when the user activity window 210 advances forward, the length of user activity window 210 remains unchanged as new activities taken from orchestration window 215 replace the activities that are either completed or dismissed by the user. As with initial activities 1-5, new activities that populate user activity window 210 over time are or become static once transferred from orchestration window 215.

Orchestration window 215, unlike activity window 210, is not re-populated with new activities after every step in sequence 200 to replace activities 6-10 that are shifted out. Thus, in this example, the length of orchestration window 215 is reduced by one at each time step t=k+1 and t=k+2 from its initial length at time t=k. However, at time t=k+3, after user activity window 210 has again advanced by one activity, orchestration window 215, unlike in previous time steps, is re-populated by performing a new determination of activities 9-13 as described herein that are computed to provide optimal value to the user.

As illustrated, in this example, activities 9-10 are the same as determined initially at time t=k, either because activities 9-10 were excluded from the determination at time t=k+3 or, alternatively, because the new determination produced the same outcome as before at time t=k. New activities 11-13 are additionally determined for the first time at t=k+3 and combined within activities 9-10. But, in general, the orchestration window 215 can be re-populated either partially in its entirety at any time step by computing a new optimal sequence of activities for the user, determined as of the current time step, using the methods and approaches as described herein.

### Next Best Engagement

Health programs can be designed so that each activity included as part of a program, if completed, will confer some health benefit to the user, where the value conferred can be represented by an abstract value that is modelled either numerically or mathematically. In some cases, the conferred health value can be modelled as an absolute value or, alternatively, as a relative score, i.e., to provide an implicit comparison with other activities. Taking into account that activities recommended to the user may not always be completed, each activity can also be associated with an expected value to the user computed as the representation of actual value to be derived from completion of the activity scaled by a probability factor that the user will in fact complete the activity, which can be trained or modelled from input data sets.

Each possible sequence of activities in a health program therefore corresponds to an overall expected health value that can be computed, for example, as the aggregate sum of the expected health values for each individual activity included in that given sequence. Because each possible activity may generally confer a different expected health value for the user, the aggregated health value to be expected from each possible sequence may also generally be unequal. Individual activities can then be recommended to the user by selecting, from among all possible sequences that may be included as part of a health program, the unique sequence that is expected to confer the greatest aggregate health value for the user.

Consider the example health program modelled by DAG 100 In FIG. 1. Assume initially that each node 105a,105b,105c,105d,105e is associated with only a single activity, i.e., Such configuration of DAG 100 defines three unique paths corresponding to different sequences of activities a user could be presented with as part of a health program. These are P₁={a₁, b₁, d₁}, P₂={a₁,b₁,e₁}, and P₃={a₁,c₁,e₁}, where a₁, b₁, etc. correspond to different possible activities.

To each path P1, P2, P3 through DAG 100, we would like to calculate the expected value for the user from suggesting the corresponding particular sequence of activities. As noted, the expected value for each path overall is equal to the sum of the expected values associated with the individual activities in the path. Of the three paths P₁, P₂, P₃ through DAG 100, the optimal path will be the one that confers the largest expected value on the user. This path may generally be selected and recommended to the user as part of a health program.

Now consider a scenario when the health program being modelled by DAG 100 has three activities c₁, c₂, c_{c} associated with node 105c as opposed to a single activity c¹. In addition to paths P₁, P₂, P₃, this configuration of DAG 100 defines two additional paths P₄={a₁,c₂,e₁} and P₅={a₁,c₃,e₁} for a total of five overall. As before, the optimal path through DAG 100 that may be selected and recommended to the user will be the one that confers the largest expected value overall.

These two example scenarios are for purposes of illustration only. In general, the process of computing and selecting an optimal path of activities may be the same for any configuration of nodes and edges in a DAG and for any number of activities associated with each node. Where a user is enrolled in multiple different health programs concurrently, each health program may be modelled by a corresponding DAG 100 and the calculation of optimal path for a single DAG 100 can generally be extended to compute a set of optimal paths through each corresponding DAG 100 used to model a health program in which the user is enrolled. The optimal set of paths in such case may be the combination of two or more paths that maximize aggregate value for the user.

Calculating a user's optimal path or paths introduces various computational dimensions. For example, antecedent user behaviours or actions may affect both the probability of a user completing a subsequent activity (e.g., because activity completion may be a reflection of user engagement with the program) and the value to be derived from its completion (e.g., because a causal relationship exists between two activities, such as stretching beforehand increasing the user's performance level in a certain exercise). Additionally, these relationships may exist across multiple different health programs in which the user is enrolled if, for example, completion by the user of an activity in one health program would impact the probability of completing another activity in a different health program and/or the value to be derived from its completion.

### User Engagement Model

The outcome we are generating is a determination of the set of activities that will be recommended as part of a user's overall plan as computed based on maximum expected value to the user. In some cases, the computation of recommended activities will be constrained by the sizes of the user activity and orchestration windows, which introduces parameters that can be controlled as a way to reduce computational effort. In general, activities can be added to a user's plan from within a single health program or across multiple health programs in which the user is concurrently enrolled or, in some cases, activities that are not associated with any program. A generalized approach involves taking each of these possibilities into consideration.

Suppose that across all programs there are m unique activities that can be recommended to users and n−1 unique programs in which users can be enrolled. Each of the m unique activities may be included in any one, or none, of the n−1 unique programs. To quantitatively represent which activities have been incorporated into a user's plan as part of which programs, let A(k) be a matrix with dimensions m×n, wherein

$${A}_{ij}^{( k )} = \{ \begin{matrix}1 & \begin{matrix}if activity i in program j is \\ included in the user ’ s plan on day k\end{matrix} \\ 0 & otherwise\end{matrix} , \qquad ( 1 )$$

for each of the first n−1 columns. Further, let column n of matrix A represent all activities in a user's plan that are not incorporated into any programs represented in the other n−1 columns of A. As defined, matrix A^{(k)} represents the set of activities (without ordering) that will be presented to a user. Matrix A^{(k)} is initially unknown and will be solved for some day, k. A solution to A^{(k)} can include both activities that will be shown to the user in the user activity window and activities that will for the time being be hidden from the user in the orchestration window.

Because antecedent user actions generally affect subsequent outcomes, we will track what the user does on each given day, k, when presented with activities from A^{(k)} in the user activity window. Let B^{(k)} be a second matrix with dimensions m×n, wherein

$${B}_{ij}^{( k )} = \{ \begin{matrix}1 & \begin{matrix}if the user completed activity i from \\ program j on day k\end{matrix} \\ 0 & \begin{matrix}if they didn ’ t do anything for activity \\ i from program j on day k\end{matrix} \\ - 1 & \begin{matrix}if the user dismissed activity \\ i from program j on day k\end{matrix}\end{matrix} . \qquad ( 2 )$$

While A^{(k)} tracks which activities are presented to the user on any given day k, matrix B^{(k)} tracks the outcome of each such activity being presented to the user. Possible outcomes can include, without limitation, completion of the activity by the user, dismissal of the activity by the user without completion, and leaving the activity pending until day k+1 (because it was neither completed nor dismissed).

Depending on how the n−1 unique programs have been authored, it is possible that a user enrolled in one or more programs may not have any activities assigned to complete on a given day, k, i.e., A^{(k)} may be null for some value k. To account for this possibility, let C^{(k)} be a vector of length n−1, wherein

$${C}_{j}^{( k )} = \{ \begin{matrix}1 & if the user is enrolled in program j on day k \\ 0 & otherwise\end{matrix} . \qquad ( 3 )$$

and is used to track, as a separate object, which of the n−1 programs a user is currently enrolled in. In general, a user may always be enrolled in at least one program, although in some cases this constraint need not be enforced.

Recall that depending on its configuration, each health program Gⱼ may define an arbitrary number of different through paths as represented by function gⱼ:Vⱼ→P({aⱼ}^{m}ᵢ₌₁). In general, the likelihood or probability of a user completing a given activity on day k, and therefore also the expected health value for the user from being presented with the activity, will vary depending on the order in which activities are presented, i.e., different expected values will be realized from different paths. We will therefore capture the ordering of activities using a permutation matrix P^{(k)} of size m×m, wherein each different state of P^{(k)} represents a corresponding ordering of activities on day k for a given solution to A^{(k)}.

For convenience, we further define

ψ^{(k)}:=(P^{(k)},A^{(k)},C^{(k)}) (4)

to represent a particular sequence of activities across all programs that may be presented to a user on each day k, and

Ψ^{(k)}:=(P^{(k)},A^{(k)},B^{(k)},C^{(k)}) (5)

to represent the set of all possible sequence outcomes for each day k of the user being presented with a given activity sequence ψ^{(k)}. Activity sequence outcomes ψ^{(k)} can include, for example, any combination and associated ordering of the user completing activities, dismissing activities from the user activity window without completing them, and leaving activities pending until day k+1.

### User Demographics

In addition to historical user behaviour, such as past engagement with or completion of activities, the probability of individual activity outcomes B^{(k)} being realized can depend on characteristics of the user itself. Let D be a vector describing static data about a user including their demographic data (such as age, gender), health risk data (such as medical conditions), and other information. Note that in general the value of D can change over time in line with user demographic data changes. For example, a user may update their marital status, their weight, age, or medical conditions.

Assumption 1 (Static User Data Assumption). In our model, we will ignore the effect of a changing vector D and assume that the likelihood of a user completing an activity depends only on the current value of D and not on other past values of D. With this assumption in place, we will ignore the effect that past values of D may have on the likelihood of activity completion and will therefore neither track nor store past values of D nor take them into account in predicting future user behaviour. Computations of expected health value may instead be performed only using the current value of D.

### Engagement Probability Distribution

Let π be a probability distribution that models the likelihood of a user on day k generating a particular outcome B^{(k)}, given activities A^{(k)} that are recommended to the user, the particular ordering P^{(k)} of those activities, the user's static demographic data D, and the user's historical engagement with activities prior to day k. The probability distribution π can be either estimated or modelled from observed data sets and will be used as an input to the computation of expected value to be derived from presenting the user an activity. In general, any probability distribution π that models the likelihood of a user generating different activity outcomes may be used. For example, different input factors (e.g., demographic data D, historical user engagement) may be taken into account, and any causal relationships between such input factors and predicted activity outcomes can be defined and/or estimated.

In general, we can expect that a user's willingness to engage with a particular activity in a particular program will be dependent on the entire past history of the user's engagement with other activities and programs. As a constraint applied to the probability distribution π, i.e., our user engagement model, we will assume that the effect of historical user behaviour is only non-zero within a defined period of time.

Assumption 2 (Markov Assumption). In terms of the engagement model, constraining the relevance of historical user engagement assumes that there is some integer N for which present outcomes B^{(k)} are independent of all past outcome sequences ψ^{(j)} under π for all j<k−N. Under this constraint, it is therefore assumed that only past outcome sequences ψ^{(j)} from within the last N days will have an impact on the likelihood of activity outcomes B^{(k)} for any given day k, and that past outcome sequences ψ^{(j)} that occurred more than N days prior to day k will have no material effect on the probability distribution π and can therefore be ignored.

### Engagement Value Function

As noted above, the amount of value derived by a user from completing a given activity is not static and may generally vary depending on the set of all activities that the user has previously completed in the past. For example, stretching prior to running may enable the user to perform at an elevated level and thereby increase the value derived from running than would have achieved without having stretched. As seen in the example, the amount of value derived by the user from the present activity differs based on the outcome of past activity activation. We will call the dependency between present value and past user actions the engagement value function, denoted V, and define it in order to capture the effect of past completed activities on the value of future ones.

Similar to the probability distribution π, we can expect the engagement value function V to be valued for the entirety of the user's past history of activity completion, even if the correlation between present value and past user actions may tend to weaken the longer the amount of time that has elapsed. As an additional constraint on the engagement model, we will further assume that the dependency between past user activity and present value to the user of completing subsequent activities is also only non-zero within a defined time period.

Assumption 3 (Value Function Assumption). In terms of the engagement model, constraining the relevance of historical activity completion on present value assumes that there is some integer M for which the value a user derives from present outcomes B^{(k)} is independent of past outcomes B^{(j)} for all j<k−M. Under this constraint, it is therefore assumed that only past outcomes B^{(j)} from within the last M days will have an impact on the value of present outcomes B^{(k)} for any given day k, and that past outcomes B^{(j)} that occurred more than M days prior to day k will have no material effect on the engagement value function V and can therefore be ignored.

The engagement value function V is another input to the computation of expected value to be derived from presenting the user with different activities and can be either estimated or modelled from observed data sets. We define the engagement value function that users derive from completing activities to be

V:X_{D}× [symbol] ^{M}→ [symbol] (6)

where B is the space of all matrices of the form (2), M represents the number of days back within which a user's past completion of activities are assumed to impact the value of completing activities in the present, and X_{D} is the static space in which user demographic data D is defined.

### Discount Factor

In the computation of expected value based on the engagement model, we also introduce a discount factor r∈R₊, which we will use to discount future values of the engagement value function V. Use of a discount factor r can be introduced in order to model the time-dependency of activity values and account for the fact that activities have increasingly less impact or benefit to present health the further into the future the activity is completed. For example, beginning a running or stretching regimen 3 months from now may be inherently more valuable and beneficial to health than 3 years from now. lessen or diminish the further into the future the activity will be completed. Any positive real number that models this effect may be selected for discount factor r.

### Next Best Engagement Optimization

Activity matrix A^{(k)} and permutation matrix P^{(k)} jointly describe the space of all possible sequences of activities that may be presented to a user on day k based on programs C^{(k)} in which the user is currently enrolled. Within this space, there generally exists a unique sequence of activities, i.e., a joint solution for A^{(k)} and P^{(k)}, which will maximize expected value for the user on day k from their engagement with health programs, given the probability distribution π of the user completing activities and the engagement value function V from completing activities. In this computation, π and V are inputs that are either first determined numerically from observed data sets or otherwise estimated or approximated and are treated as constants. Some example probability distributions π and engagement value functions V are described in more detail below. As noted, a suitably chosen discount factor r for future activities can also optionally be introduced to the model.

Mathematically, the computation of expected value represents the solution to the following optimization problem, given π, V, and optionally r:

$${arg max}_{( {ψ}^{( k )} , {ψ}^{( k + 1 )} , … )} {\sum }_{( {B}^{( k )} , {B}^{( k + 1 )} , … )} {\sum }_{d = 0}^{∞} π ( {B}^{( k + d )} \vert 𝒟 , {Ψ}^{( k + d , … , N )} , … , {Ψ}^{( k + d - 1 )} , {ψ}^{( k + d )} ) \frac{V ( 𝒟 , {B}^{( k + d … M )} , … , {B}^{( k + d )} )}{{( 1 + r )}^{d}} \qquad ( 7 )$$

In the above equation, the inner summation computes the aggregate expected value of presenting the user with the sequence of activities associated with a given path through health program Gⱼ, and the outer summation performs this computation over all possible paths or activity sequences that are defined for health program Gⱼ. The solution will be the particular values of A^{(k)} and P^{(k)} that jointly maximizes the argument, i.e., that produces the largest aggregated expected value for the user associated with an activity sequence.

In the computation of the inner summation, index d starts at d=0 corresponding to the current day, k, and is generally unbounded to allow for a health program Gⱼ that is of any arbitrary length. Introduction of the discount factor r to the value of future activities, which grows at an exponential rate as a function of time, can facilitate convergence of the inner summation by driving down the value of future activities asymptotically toward zero (the inner summation is otherwise infinite and not guaranteed to converge). In this manner, use of a discount factor r does not just model the diminishing value of health activities into the future, but also represents a useful constraint on computational effort at the same time.

Solutions for A^{(k)} and P^{(k)} can be determined with equation (7) over the entire length of the health program Gⱼ or, by limiting the range of index d in the inner summation, to any other arbitrary length of time. For example, as described herein, equation (7) can be solved on day k to determine solutions for matrices A^{(k)} and P^{(k)} only within the user activity and orchestration windows, which may generally have a combined chosen length that is shorter than the length of health program Gⱼ overall, by selecting an appropriate range of values for index d. Doing so can impose a useful constraint on equation (7) that reduces the computational effort involved in generating optimal solutions to matrices A^{(k)} and P^{(k)}.

Excluding the discount factor r, the inner argument in equation (7) computes expected value for the user as the product of probability distribution π and the engagement value function V valued for each day t=k, k+1, k+2, . . . in the inner summation. With the discount factor r introduced, future values of the engagement value function V decrease exponentially toward zero. As described herein, the probability distribution π is modeled as the conditional probability of each possible outcome B^{(k)} occurring given a present sequence of activities ψ^{(k)} representing a particular path through health program Gⱼ that is or could be recommended to the user, the set of past activity outcome sequences ψ^{(k-N)}, . . . , ψ^{(k-1)} observed over the previous N days (through application of the Markov assumption), and the current state of the user demographics vector D. The engagement value function V is additionally valued for each possible outcome B^{(k)} given the current state of the user demographics vector D and the set of past activity outcomes ψ^{(k-M)}, . . . , ψ^{(k-1)} observed over the previous M days (through application of the Value Function assumption). Where the probability distribution π and engagement value function V are finitely valued, a non zero discount factor r tends to drive convergence of the inner summation in equation (7).

Additional constraints may be imposed on equation (7) to meet different use cases, including to model different parameters or characteristics of health program G. For example, any one or more of the following constraints may be imposed in different embodiments:
1. Activities are only included or excluded. That is, Aᵢⱼ^{(k)}∈{0,1} for all i, j, k.
2. Activities are only completed, dismissed, or carried over. That is, Bᵢⱼ^{(k)}∈{−1,0,1} for all i, j, k.
3. All orderings of activities are possible. That is, P^{(k)} is a matrix of dimension m×m for all k.
4. Excluded activities can be neither completed nor dismissed. That is, if Aᵢⱼ^{(k)}=0, then Bᵢⱼ^{(k)}=0.
5. Included activities that are neither completed nor dismissed are carried over. That is, if Aᵢⱼ^{(k)}=1 and Bᵢⱼ^{(k)}=0, then if Aᵢⱼ^{(k+1)}=1.
6. Activities from non-enrolled programs are excluded. That is, if Cⱼ^{(k)}=0, then A_{q}^{(k)}=0 for all activities i in program j.
7. Program DAGs are followed. That is, if Cⱼ^{(k)}=1, Aᵢⱼ^{(k)}=1, and Bᵢⱼ^{(k)}≠0, then Aᵢⱼ^{(k+1)} must be consistent with Gⱼ for all activities i included in program j.
8. Finite-length user activity (or orchestration) window is applied. That is, ∥A^{(k)}∥≤N, for some fixed N where ∥A^{(k)}∥²:=A^{(k)}.A^{(k)} defines the number of activities presented to the user in the activity window (or alternatively sequenced for presentation to the user in the orchestration window) on each day k.

### Optimizing for Engagement Over Value

In general, the engagement value function V can be differently valued over all activities and historical use cases so that equation (7) optimizes for expected value to the user of being presented with activities. As an alternative to optimization for value, equation (7) can be adapted to optimize instead for user engagement by assuming that all activities will confer equal value to the user in all circumstances if completed. We will call this the equal value assumption.

Assumption 4 (Equal Value Assumption). By fixing the value engagement function to V=1 for all B^{(k)}, . . . , B^{(k-m)}, we are assuming that all activities have the same value for the user in all use cases. With this constraint in place, equation (7) searches for the joint solution to A^{(k)} and P^{(k)} representing the sequence of activities with which the user is expected to have the highest overall engagement, i.e. containing the most number of activities that the user will complete. When no activity is valued more than another, the greatest user benefit is achieved by maximizing the number of completed activities of any kind, which effectively serves as a measure of user engagement.

### Program and Activity Independence

Independently of the value engagement function V, different models for the probability distribution π can also be constructed and used as an input into equation (7) to meet different use cases and/or account for different causal relationships. Like the value engagement function V, this may take the form of additional constraints or assumptions imposed on the probability distribution π, such as to model different user behaviour or for any other purpose. Equation (7) can operate on any suitable model of the probability function π however defined or constrained (in addition to different formulations of an engagement value function V).

For example, while historical user behaviour may generally affect the probability of a user completing activities in the present or future, effectively modelling these relationships may be impractical without having first observed large and potentially complex data sets. Imposing some additional assumptions about user behaviour can allow us to define a modified probability function π* that is constructed from smaller and/or less complex data sets. Such a modified probability function π* can usefully approximate the probability function π in which historical user behaviour is more completely modeled.

Assumption 5 (Program Independence Assumption). As between any two health programs, let it be assumed that the probability of user engagement with one program is independent of the other, whether the user is currently enrolled in or has in the past enrolled in either or the two programs. Under this assumption, the likelihood of the user enrolling in one of the programs does not change if the user enrolls or has enrolled in the other program. More generally, the likelihood of the user enrolling in any given program on day k has no dependency on whether or not the user has enrolled in any other past or present program.

Note that the program independence assumption does not imply every program has the same probability of enrollment by the user, but rather that preference for one program will not be taken to affect preference for another. To illustrate, assume that the user is 40% likely to enroll in a running program and 30% likely to enroll in a nutrition program. Under the program independence assumption, the user remains 40% likely to enroll in the running program notwithstanding present or past enrolment in the nutrition program, which might otherwise have translated to an increased likelihood of the user also engaging with the running program being modeled. User preference for each particular program may be estimated, for example, taking into account the user demographic data D.

Assumption 6 (Activity Equivalence Assumption). Within a given program, let it also be assumed that the user has equal probability of completing any available combination of p activities within a program. Under this assumption, user engagement with activities is only modeled in terms of the overall number of activities that are completed and not on which activities in particular are or aren't completed. In terms of contribution to user engagement, activities are treated as equivalent and therefore indistinguishable from one another from the perspective of user engagement.

Note that the activity equivalence assumption does not imply an equal probability of completing different numbers of activities. For example, consider a program consisting of 3 different activities. Under the activity equivalence assumption, the user is taken to have the same probability of completing either activities 1 & 2 or activities 1 & 3 or activities 2 & 3. However, the probability of the user completing all 3 activities or only a single one or none of the activities may generally all be different because the user's likely or predicted level of engagement will still depend on demographic factors.

In some cases, as an additional constraint, it may be convenient or expedient in some circumstances to order recommended programs (or their constituent activities) for the user sequentially based on when the user enrolled in the program. Sequential ordering may be selected over alternative orderings of activities or programs for the user as computed to maximize expected value or user engagement, even though sequential order may confer less than maximum expected value or engagement. For example, it may be convenient to impose sequential ordering of activities in conjunction with one or both of the program independence and activity equivalence assumptions. In such cases, P^{(j)} may be ignored when modelling the probability function π.

Under the program independence and activity equivalence assumptions, and applying further the constraint that activities will be presented sequentially in order of when the user enrolled in each active program, in which case past activity orderings P^{(j)} of activities can be ignored, equation (7) can be rewritten as:

$${\sum }_{c ∈ {C}^{( k )}} {\sum }_{0 \leq k \leq \vert c \vert} \frac{\begin{matrix}\tilde{π} ( User completes k activities \\ from program c \vert 𝒟 )\end{matrix}}{( \begin{matrix}\vert c \vert \\ k\end{matrix} )} \{ ( \begin{matrix}\vert c \vert - 1 \\ k - 1\end{matrix} ) {\sum }_{l = 0}^{\vert c \vert - 1} \frac{1}{{( 1 + r )}^{l}} \} \qquad ( 8 )$$

where π* represents a probability distribution that models the number of activities a user is likely to complete within a given program. Note that probability distribution π* in equation (8) represents a different model of user behaviour compared to the probability distribution function π used as an input to equation (7).

Equation (8) computes user engagement as the conditional probability of the user completing a given number of activities within a program given user demographics D, averaged over the total number of ways to choose that number of activities as part of the program, and multiplied by an aggregate measure of discounted value or engagement over all combinations of the given number of activities. For example, in an example program consisting of 4 different activities, there are 6 different ways to choose any 2 activities. The probability of the user completing any 2 activities is spread evenly over all 6 individual combinations. The aggregate measure of discounted value in equation (8) counts the total value to the user of all possible combinations of the given number of activities taking into account the position of each activity within the possible sequence and discounted to the present by the appropriate multiplier.

Since the summands in equation (8) are independent of one another, each term can be separately estimated. This will generate an engagement score for each program individually that can be used to rank and recommend programs to users based on expected relative engagement.

Matrix Factorization with Specification

To estimate the modified probability distribution function π* in equation (8), which models the likelihood of a user completing different numbers of activities within a given program, an approach based on matrix factorization can be utilized. In matrix factorization, recommendations can be generated by discovering a number of “latent” qualities of users that show certain behavioural preference based on observed data in order to make predictions of similar behavioural preference that has not been directly observed in other users. A significant limitation of matrix factorization is that the “latent” qualities used in the prediction generally have no interpretable meaning or don't correlate to explicit user characteristics, such as age, gender, known health risks, etc. Further, there is generally not much control over how these latest qualities are defined; rather than being selected, they are discovered by the method itself. We will define a modified form of matrix factorization that addresses these limitations.

Suppose now that we have m different programs that can be presented to n different users. Additionally let there be a ratings matrix R of dimension n×m where each element represents a measure of preference that the user u has exhibited or which has been observed toward health program c. In our assumption, not every user u will have completed every health program c and therefore not all preferences or ratings will be known. Ratings matrix may in general be incomplete and in some cases may be relatively sparsely populated.

In our modified approach, let P be a matrix of dimensions n×(k+l) and Q be a matrix of dimensions m×(k+l). In each case, k will be chosen to represent the number of latent qualities that will be discovered, while l represents the number of explicit user features that we have chosen to include in our model (e.g. age, gender, health risks, etc.). In general, these features may be known for some users but unknown for other users. We would like to find solutions for P and Q whose matrix product best approximates ratings matrix R_{u,c}. To do this, we can solve values for P and Q that minimizes the cost function

$$ℒ = {\sum }_{u , c} {( {R}_{u , c} - {P}_{u , :} {Q}_{c , :}^{T} )}^{2} \qquad ( 9 )$$

where Pᵢⱼ=xᵢ^{(u)} if feature 1≤i≤1 for user u is known, and P_{u,j}=0 if feature 1≤i≤l is unknown for user u. The remaining undefined entries in P and Q are then solved by minimizing the cost function.

We can adapt ratings matrix R_{u,c} to estimate of the modified probability distribution function π* in equation (8). We begin by defining ratings matrix R_{u,c} so that each individual rating is either a 0 or 1 corresponding to whether or not the user met or exceeded a certain target, however defined, which can in general be based on any suitable threshold or criteria to be satisfied. An entry of 0 in ratings matrix R_{u,c} indicates that user u has not met or exceeded the target in relation to program c, and an entry of 1 in ratings matrix R_{u,c} indicates that the target has been met or exceeded. With ratings matrix R_{u,c} defined in this way, the solution to P and Q then provides a numerical score for every user u and every program c. We interpret this score as the likelihood or probability that user u would also meet or exceed the target in relation to program c.

With ratings matrix R_{u,c} thus defined to include only 0s or 1s, a modified cost function can also be introduced in the form of:

$$ℒ = - {\sum }_{u , c} {R}_{u , c} log g ( {P}_{u , :} {Q}_{c , :}^{T} ) + ( 1 - {R}_{u , c} ) log ( 1 - g ( {P}_{u , :} {Q}_{c , :}^{T} ) ) \qquad ( 10 )$$

where g(x) in equation (10) is the sigmoid function. As before, P_{i,j}=xᵢ^{(u)} if feature 1≤i≤l is known for user u and P_{u,j}=0 if feature 1≤i≤l is unknown for user u, where xᵢ^{(u)} can be defined as any real number 0≤xᵢ^{(u)}≤1 that represents feature i of the user u. Additionally, the solution to equation (10) can be generally constrained so that:

0≤P_{u,i}≤1

0≤Q_{u,i}≤1

for all 1≤i≤l corresponding to the explicitly chosen user features.

To estimate the modified probability distribution function π* in equation (8), we can define a corresponding ratings matrix R_{u,c} in terms of an appropriate target for each scenario of user behaviour to be modelled. In general, to model the probability of a user completing a given number of activities within a program, a ratings matrix R_{u,c} can be constructed for that program that tracks, as the specified target, whether or not users who enrolled previously in that program completed at least that number of activities, i.e., whether or not such users met or exceeded the target number. As many such ratings matrix R_{u,c} can then be defined as there are activities in the program in order to track a plurality of different activity completion rates simultaneously. Corresponding solutions to P and Q that are determined by minimizing equation (10) then provide probability estimates for inclusion in the modified probability distribution function π*.

Consider again an example program consisting of 4 activities. The probability of a user completing exactly one activity can be estimated by defining a ratings matrix R_{u,c} that tracks whether users u that enrolled in program c reported or were observed to have completed that number of activities. Additional ratings matrices R_{u,c} can likewise be defined for completion of any 2 activities, any 3 activities, or all 4 activities. Solutions to P and Q obtained from equation (10) then together provide probability estimates for any given user u completing different numbers of activities in program c for all users u across all programs c.

In alternative embodiments, ratings matrix R_{u,c} can be defined to track whether or not a target percentage of activities have been completed, as opposed to exact numbers of activities that have been completed by a user. In some cases, entries for each ratings matrix can be either a 0 (indicating that target percentage was not achieved) or 1 indicating that the target was achieved. For example, a ratings matrix can be defined to track whether or not a user completed 50% of the activities within a program. But, in general, any number of such ratings matrices can be defined to track different target ranges, such as 0-25%, 25-50%, 50-75%, and 75-100% of activities, and so on, as one example.

Tracking percentages of activities completed in ratings matrix as opposed to numbers of activities, may be useful where programs c contain generally different numbers of activities. By estimating the likelihood of a user completing a certain percentage of activities generally, this can be transformed on a program by program basis into an estimate of the probability of completing a certain number of activities based on the total number of activities in the program.

### System Architecture

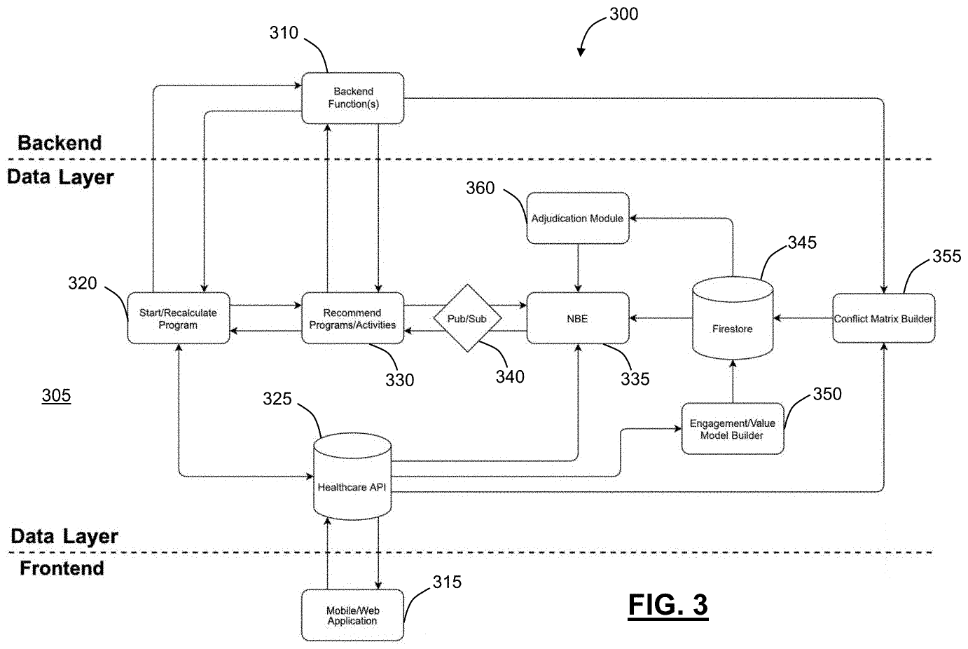

Referring now to FIG. 3, there is shown an example data processing system 300 that may be suitable for recommending activities or programs to users in accordance with the disclosed embodiments. System 300 may generally comprise a plurality of different modules or data processing components that are implemented within a data processing layer 305 located between and that interfaces with each of backend function(s) 310 and a frontend user application 315 that may be, for example, a mobile or web-based computer application.

In accordance with the disclosed embodiments, system 300 may generally be configured to provide users with dynamically generated recommendations of health programs or activities (more generally “health and wellness content”) for the user to complete. Recommended programs or activities can be computed by system 300 based on user demographic information and historical user behaviour using any of the processes, methods, and algorithms described herein so as to maximize expected health value or engagement of the user from being presented with such activities or programs.

As shown, data processing layer 305 in system 300 may for example include a start/recalculate program module 320 in communication with backend function(s) 310, which can for example include health program authoring and other content generation as described herein. Start/recalculate program module 320 may further communicate with a healthcare application program interface (API) 325 that exposes data processing layer 305 to mobile/web application 315, which can be installed on a user device, such as a mobile phone, tablet, portable or desktop computer, or smart watch as well as others, for example. Information and data exchange between healthcare API 325 and mobile/web application 315 may be bilateral, i.e., mobile/web application 315 may both provide to and receive data from data processing layer 305 through healthcare API 325.

In some embodiments, start/recalculate program module 320 may be configured to deliver health programs to users by pushing health content to mobile/web applications 315 installed on user devices. As described herein, health programs may be delivered to users as sequences of recommended activities that are displayed or framed for the user with use of a user activity window. Activities or activity sequences for start/recalculate program module 320 to push to users of mobile/web applications 315 may be provided or retrieved from a recommend program/activities module 330 whenever new programs or activities are to be recommended.

When a user is commencing a new health program, start/recalculate program module 320 may request or retrieve an initial sequence of recommended activities from recommend program/activities module 330 as defined by a user activity window. Thereafter, as a user completes activities in the program over time, start/recalculate program module 320 may periodically update the user activity window in mobile/web application 315 with additional and/or updated activities provided by recommend program/activities module 330 until program completion. In some cases, users of mobile/web application 315 may expressly request that programs and/or activities be recommended. Alternatively, recommendations may originate automatically from within data processing layer 305, such as from start/recalculate program module 320 or from recommend program/activities module 330.

In order to provide recommendations of programs or activities for users, recommend program/activities module 330 can be configured to call or invoke a next best engagement (NBE) engine 335 via a publication/subscription (pub/sub) interface 340. Recommend program/activities module 330 may either automatically or upon request invoke NBE engine 335 to return a set or sequence of activities that are computed by NBE engine 335 for a given user using any one or more of the processes as described herein so as to maximize that user's expected health value or program engagement. For example, NBE engine 335 may determine activities that will maximize expected health value for a user by solving a form of equation (7) or, alternatively, activities that will maximize expected user engagement by solving a form of equation (8) under different program constraints or to meet different use cases without limitation.

The number of activities returned by the NBE 335 in a sequence (or partial sequence) may be based on the combined size of the user activity and orchestration window and the user's progress through a program. For example, as explained herein, activity recommendations may initially be generated by NBE engine 335 to fill the user activity and orchestration windows and thereafter so as to periodically re-populate the orchestration window with new activities that will shift in sequence into the user activity window as the user progresses through a program. In general, NBE engine 335 can be invoked to generate activity recommendations with a sufficient frequency that avoids the orchestration window from being completely emptied of activities.

Input data for the NBE engine 335 to compute optimized activity sequences may be stored in a suitable database, such as firestore 345, which is accessible to NBE engine 335. For example, firestore 345 may store data sets containing user demographic information (e.g., age, sex, health conditions), in the form of a corresponding vector D, which has been supplied by the user to mobile/web application 315. Firestore 345 may additionally store one or more different probability functions π or π* and one or more different engagement value functions V that have been modeled or constructed by engagement/value model builder 350 from observed historical user behaviour combined with user demographic information.

Engagement/value model builder 350 may receive and process input data sets, e.g., containing observed historical user behaviour or user demographic information supplied by the user to mobile/web application 315, into applicable models of probability functions π or π* or engagement value functions V. For example, a modified probability function π* may be constructed by engagement/value model builder 350 defining and applying matrix factorization to a plurality of suitably defined ratings matrices as described herein.

In some embodiments, system 300 may additionally include a conflict matrix builder 355 and adjudication module 360. Conflict matrix builder 355 may be configured to pre-compute or determine activity conflicts based on health program data received from backend function(s) 310 and/or user demographics or historical user engagement data supplied to or observed on mobile/web application 315. Activity conflicts may be defined to include any activity that is determined to be incompatible or otherwise inconsistent with another activity from a different program. For example, the two activities of running and resting would be incompatible if recommended to the user on the same day. Two activities within a given program may also conflict (although in general health programs can be authored within backend function(s) 310 to avoid any inherent activity conflicts). Additionally, activity conflicts may be defined to include any activity that is determined to be incompatible or inconsistent with a particular user characteristic or trait (such as might occur if certain activities were to be recommended to an individual with a physical disability).

Given a set of health programs authored on backend function(s) 310, conflict matrix builder 355 may generate a matrix of activity conflicts for each different user within system 300. Each resulting activity conflict matrix may be stored, for example, in firestore 345 for use by adjudication module 360 as an additional input into NBE engine 335. In some cases, for example, adjudication module 360 may transform an activity conflict matrix retrieved from firestore 345 corresponding to a particular user into additional rules or constraints that NBE engine 335 will impose on the computation of optimal solutions for k^{(x)} and P^{(x)} computed by equation (7) or equation (8). Such rules could involve sequencing two activities in a particular order that resolves or avoids a conflict, restricting the inclusion of an activity if a conflict activity has already been or will be recommended, restricting the inclusion of an activity based on a conflict with user demographic vector D, and so on. In general, the output from NBE 335 may be computed in a way that optimizes expected value for the user or program engagement without introducing or by prioritizing the avoidance of any activity conflicts.

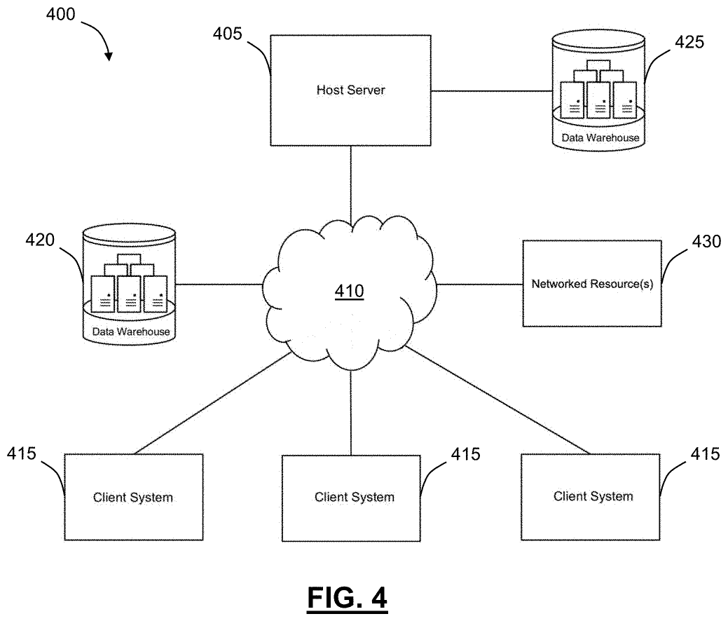

FIG. 4 illustrates an example system implementation 400 of data processing system 300 that is based on a server-client architecture in accordance with the embodiments disclosed herein. As shown, system 400 may include a host server 405 that is connected to a network 410, which can be any local, wide area, or mobile network, including the Internet, cellular networks, and the like, or any combination of the foregoing. A plurality of client systems 415 may be in communication with host server 405 through network 410 and used by individual users to access services that are available on host server 405. An example of three client systems are shown in FIG. 4, but in general there may be any number n of client systems 415 corresponding to n unique users registered within data processing system 300 as described herein.

In some cases, host server 405 may be or include any local or on-premises computer system configured with hardware and/or software resources that allow local or remote access by client systems 415. Alternatively, host server 405 may instead be implemented on a distributed or cloud-based environment, such as Google™ Cloud Services, Amazon™ Web Service, or Microsoft™ Azure™ Cloud Computing Platform, which may be located remotely at one or more third party data centres. A cloud-based implementation of host server 405 may advantageously facilitate scalability in respect of storage memory or computational requirements compared to a self-hosted alternative.

Host server 405 provides a computing and data processing platform that is operable to coordinate the operation of data processing system 300 and deliver health content to client devices 415 across network 410. Data processing layer 305 of system 300 can be implemented using or including a client-server configuration in which the exchange of data between host server 405 and client systems 415 is controlled using remote procedure calls and API-level communication. For example, healthcare API 325 of data layer 305 within data processing suite 300 may be used to communicate with a corresponding web/mobile applications 315 that are implemented on client systems 415. Host server 405 may also incorporate encryption, such as SSL or other suitable security protocols, to secure communication and exchange of confidential and/or personal information with client devices 415.

One or more data warehouses or databases can be implemented within system 400 including, for example, a networked data warehouse 420 that is accessible to host server 405 over network 410 and/or a local data warehouse 425 that is dedicated to host server 405 directly. Each data warehouse 420, 425 can be any suitable type of data storage system, such as a memory, disk, or monolithic or distributed database, which is configured to store information and data sets that are processed within data processing layer 305 as described herein. For example, firestore 345 may be implemented on any combination of suitably configured data warehouses 420, 425.

In some cases, data warehouses 420,425 may be or include any local or on-premises computer system that is network-enabled or otherwise in communication with host server 405. Alternatively, either or both of data warehouses 420, 425 may also be implemented on a distributed or cloud-based environment, such as Google™ Cloud Services, Amazon™ Web Service, or Microsoft™ Azure™ Cloud Computing Platform, which may be located remotely at one or more third party data centres.

The content items stored within data warehouse(s) 420,425 may be associated with a suitable identifier including information identifying an entity or content type. For example, each of the m health programs stored within data warehouse(s) 420,425 can be assigned a corresponding health program item ID as well as one or more tags corresponding to different program parameters and other information. Additionally, each of the n users may have stored within data warehouse(s) 420,425 a corresponding health profile that contains demographic information for the user, which may be either static or updated from time to time by the user.

For example, as the user engages with system 400 and is recommended activities or programs, the user's particular sequence of actions, such as whether a given program or activity was completed, can be captured on client devices 415 and recorded to the user's respective health profile maintained within data warehouse(s) 420,425. Each user health profile can be updated over time to indicate the user's progress through a recommended health program as well as other data or metrics relating to activity completion, such as time, calorie expenditure, speeds, etc. User progress through a health program may be tracked using one or more different methods such as self-reporting and/or via the use of wearable telemetric devices such as smart phones and smart watches.

User health profiles may in some cases be initially only sparsely populated with certain static information about the user, such as the individual's name, address, date of birth, etc. Subsequently, or gradually over time, user health profiles may be completed or filled out with additional demographic information that is supplied by the user to client system 415. User health profiles may be updatable using various methods, including, but not limited to, the user's interaction with system 400, periodic check-ins with the user, activity tracking via client system 315 and/or telemetric device integration, and data from external or third party partners, such as networked resource(s) 430.

User demographic information may for example be collected from a health assessment presented to the user comprising a list of questions the user is asked to complete, such as questions relating to the user's general health, medical/health history, current medications, health and wellness goals (e.g. to lose a certain amount of weight, to train for a marathon, etc.), health/lifestyle interests, and health data (e.g. weight, height, existing medical conditions), and the like. Each client system 415 may also be integrated with or coupled to various telemetric devices and platforms capable of measuring different biological indicators such as temperature, blood pressure, and heart rate, which can then be provided to client system 415 for entry into the user's health profile.

In some embodiments, in addition to or as an alternative to the health assessment, the user may authorize access to one or more networked resource(s) 430, such as external databases managed by third-party data providers, to obtain health related information. For example, the user may authorize host server 405 to contact the user's pharmacist by way of networked resource(s) 430 to obtain a list of medications the user is currently taking or has taken. The type of medication taken by the user may indicate the user's health status (i.e. existing medical conditions) and/or allow the inference of possible health-related risk factors associated with that user.

The networked environment shown in FIG. 4 that provides an example server-client implementation 400 of data processing system 300 is for illustration purposes only. Different implementations and/or architectures will be possible to fit different use cases of a data processing system 300 that generates recommendations of health-related content for users.

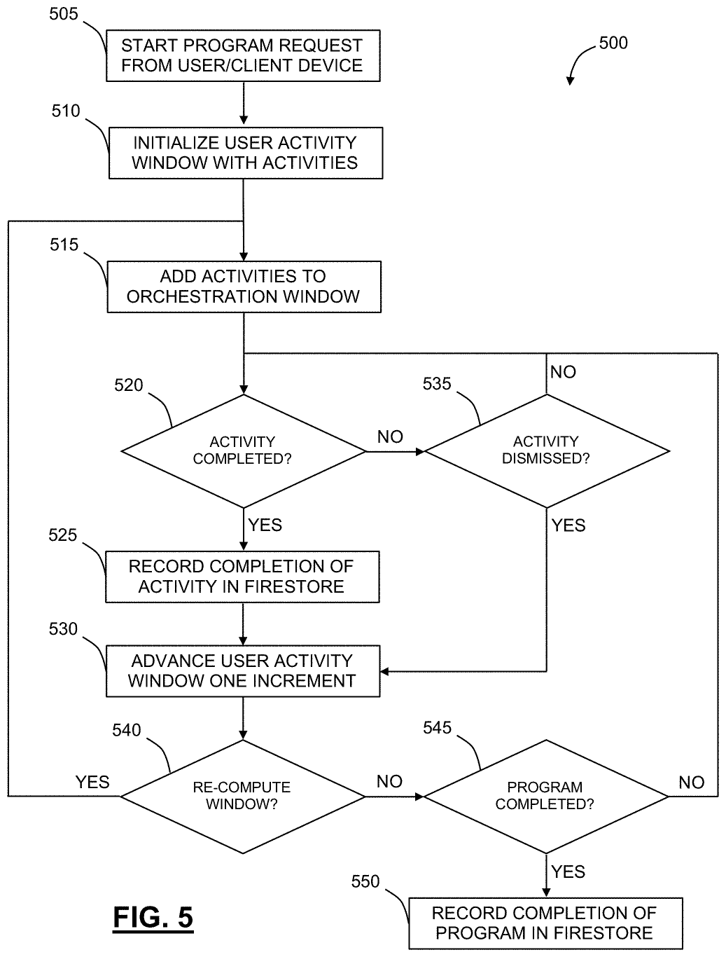

FIG. 5 illustrates an example method 500 of delivering a dynamic health program to a user device in accordance with the described embodiments. Method 500 may be performed, for example, by or within data processing layer 305 of data processing system 300 shown in FIG. 3 or on a host server 405 within the example network environment 400 shown in FIG. 4. The example implementation of method 500 shown in FIG. 5 is for illustrative purposes only. According to different implementations of method 500, some examples of which are described further below, individual steps or processes may be performed differently or in a different order than as shown, by a different component, etc., or in some case omitted entirely, while additional steps and/or sequences not illustrated in the example method 500 may also be included as described herein generally without limitation.

For example, as illustrated, method 500 may begin at 505 by data processing layer 305 or host server 405 receiving a user request from a mobile/web application 315 or client system 415 to initiate a dynamic health program. In some cases, a NBE engine 335 may compute an optimal program or ranked set of optimal programs for the user to compute based on expected value or engagement for the user as described herein, and the user request may be transmitted in response to data processing layer 305 or host server 405 automatically pushing a recommendation of one or more health programs to mobile/web application 315 or client system 415 from which the user may select one to initiate. In some cases, the user request may be generated by the user browsing from a list of all available health programs that is pushed to mobile/web application 315 or client system 415 and making a selection of a program to initiate. In some cases, the user request may be a selection from a list or subset of health programs that have been recommended to the user or which have been determined to be of interest to the user.

Following receipt at 505 of the user request to initiate a health program, a user activity window may be initialized at 510 with a sequence of activities and, at 515, optimal activities may also be added to an orchestration window. As described herein, for example, an NBE engine 335 may be configured to recommend activities for the user activity and orchestration windows that are computed based on any of the approaches described herein to optimize expected value or engagement to the user. After being presented at 510 with activities, the user may begin to progress through the health program by completing or dismissing activities in sequence until the end of the program is reached. Activities added to the orchestration window at 515 may be computed but hidden from the user until shifted into the user activity window, as described herein.

Thus, for example, method 500 may at 520 check, e.g., by periodically polling mobile/web application 315 or client system 415, whether the next activity sequenced for the user within the user activity window has been completed. If it is determined at 520 that the next activity has been completed, at 525, completion of the activity may be recorded in firestore 345 or engagement/value builder 350 to be used for generation or refinement of engagement probability models as described herein. The user activity window may then at 530 be advanced by one increment to the present the next sequenced activity to the user, and to replace the completed activity with the next computed activity from the orchestration window, which may at the same time be decremented by one activity.

If at 520 it is determined that the next sequenced activity has not been completed by the user, at 555, method 500 may check whether the next sequenced activity has been dismissed by the user without completion. If at 535 it is determined that the next sequenced activity has not been dismissed by the user either, then method 500 may loop back around to 520 to poll again whether the next sequenced activity has been completed in the time elapsed since the last check. In this way, checks 520 and 535 may be performed repeatedly until the next sequenced activity is completed or dismissed, although in some embodiments, mobile/web application 315 or client system 415 may instead push notification of activity completion or dismissal to data processing layer 305 or host server 405 instead of being repeatedly polled. If at 535 it is determined that the next sequenced activity has been dismissed by the user without completion, then method 500 may advance the user activity window at 530 without recording completion of the activity or, alternatively, by recording dismissal of the activity within firestore 345 or engagement/value builder 350.

At 540, it is determined whether to populate the orchestration window with a new set of activities for the user that are computed as described herein to optimize user value or engagement with the program. If it is determined at 540 to re-populate the orchestration window, then method 500 may loop back to 515 to add new activities to the orchestration window. The orchestration window may be re-populated entirely with new activities in some cases or, alternatively, only with activities to fill vacancies.

Repopulation of the orchestration window may occur at different frequencies or interval periods. For example, it may be determined at 540 to re-populate the orchestration window with each advancement of the user activity window (following completion or dismissal of the next sequenced activity) until the end of the program so that the orchestration window remains full of activities. Alternatively, it may be determined at 540 to re-populate the orchestration window at a slower frequency, provided that in some cases the orchestration window is not permitted to become entirely depleted of activities.

If it is determined at 540 not to populate the orchestration window, then it may be checked at 545 whether the user has reached the end of the program. If the user has reached the end of the program, then at 550 a record of completion may be added to firestore 345 or engagement/value builder 350 to be used subsequently in the generation or refinement of engagement probability models as described herein. If it is determined at 545 that the end of the program has not been reached, the method 500 may loop back to 520 to check for completion of the next sequenced activity without adding new activities to the orchestration window at 515.

As described and shown, a separate instance of method 500 and/or its alternative embodiments may be performed for each different health program that the user enrolls in or requests to initiate. In some cases, instances of method 500 may be performed one at a time in the order in which program requests are received. Alternatively, instances of method 500 may be performed concurrently where the user is enrolled in multiple programs simultaneously. Where a user is enrolled in multiple programs simultaneously, activities can be recommended to the user that optimize user health value or engagement across each program individually or across all programs jointly as described herein using a suitably defined probability distribution model and/or engagement value function.

In the example configuration shown in FIG. 5, method 500 may be initiated by a start program request being received (i.e. at 505) from a user/client device. However, in other embodiments, method 500 may instead be initiated instead by data processing layer 305 or host server 405 transmitting a health program start notification to a mobile/web application 315 or client system 415. In some cases, a single start for a corresponding health program may be transmitted or, alternatively, multiple start notifications for multiple different health programs concurrently. In some cases, multiple instances of method 500 may be performed simultaneously in which some are initiated by the user sending a start program request and others are initiated by sending a program start notification to the user. Still other variations are possible to fit different end uses or applications consistent with the described embodiments.

The examples and corresponding diagrams used herein are for illustrative purposes only. Different configurations and terminology can be used without departing from the principles expressed herein.

Although the invention has been described with reference to certain specific embodiments, various modifications thereof will be apparent to those skilled in the art without departing from the scope of the invention. The scope of the claims should not be limited by the illustrative embodiments set forth in the examples, but should be given the broadest interpretation consistent with the description as a whole.

1. A method of recommending a sequence of activities to a user, the method comprising:
determining a probability distribution that represents, for each of a plurality of different activities, an estimated conditional probability of the user completing the corresponding activity, wherein each estimated conditional probability is based on demographic data about the user and a historical user engagement function that defines a relation between past outcomes of the user being presented with different sequences of the activities and the corresponding estimated conditional probability;
defining an engagement value function that represents, for each of the plurality of different activities, a corresponding numerical value assigned to the user of completing the activity, wherein each assigned numerical value is based on the demographic data about the user and a historical activity completion function that defines a relation between past completed activities of the user and the corresponding assigned numerical value;
computing, for each of a plurality of possible activity sequences, a total expected value for the user of being presented the corresponding activity sequence, wherein
individual activities included within a possible activity sequence are associated with a corresponding expected value to the user that is computed based on the the probability distribution and the engagement value function, and
each total expected value is computed as an aggregate sum of expected values associated with the individual activities included within the possible activity sequence;
selecting, from among the plurality of possible activity sequences, a recommended sequence of activities that is determined to provide the greatest total expected value for the user; and
transmitting the recommended sequence of activities to the user.

2. The method of claim 1, wherein the expected value to the user of completing an individual activity is computed as a product of the probability distribution and the engagement value function.

3. The method of claim 2, wherein the expected value to the user of completing an individual activity is further computed by applying a discount factor to the engagement valued function that exponentially diminishes the assigned numerical value to the user in the present of activities completed in future.

4. The method of claim 1, wherein the historical user engagement function is defined over a finite period of time.

5. The method of claim 1, wherein the historical activity completion function is defined over a finite period of time.

6. The method of claim 1, wherein the plurality of possible activity sequences comprises all possible activity sequences defined for a health program in which the user is enrolled.

7. The method of claim 1, wherein the demographic data about the user is assumed to be static.

8. The method of claim 1, wherein a length of the computed recommended sequence of activities transmitted is constrained by a user activity window and an orchestration window.

9. The method of claim 8, further comprising displaying a first portion of the computed recommended sequence of activities defined by the user activity window to the user.

10. The method of claim 9, further comprising transferring a second portion of the computed recommended sequence of activities defined by the orchestration window into the user activity window for display to the user.

11. A server-implemented system for recommending a sequence of activities to a user, the system comprising:
a model builder configured to generate a probability distribution and an engagement value function
wherein the probability distribution represents, for each of a plurality of different activities, an estimated conditional probability of the user completing the corresponding activity, wherein each estimated conditional probability is based on demographic data about the user and a historical user engagement function that defines a relation between past outcomes of the user being presented with different sequences of the activities and the corresponding estimated conditional probability, and
wherein the engagement value function represents, for each of the plurality of different activities, a corresponding numerical value assigned to the user of completing the activity, wherein each assigned numerical value is based on the demographic data about the user and a historical activity completion function that defines a relation between past completed activities of the user and the corresponding assigned numerical value; and
a next best engagement (NBE) engine in electronic communication with the model builder, the NBE engine configured to generate a recommended sequence of activities for transmission to the user by:
computing, for each of a plurality of possible activity sequences, a total expected value for the user of being presented the corresponding activity sequence,
wherein individual activities included within a possible activity sequence are associated with a corresponding expected value to the user that is computed based on the the probability distribution and the engagement value function, and
wherein each total expected value is computed as an aggregate sum of expected values associated with the individual activities included within the possible activity sequence; and
selecting, as the recommended sequence of activities for transmission to the user, one of the plurality of possible activity sequences that is determined to provide the greatest total expected value for the user.

12. The system of claim 11, wherein the NBE engine is configured to compute the expected value to the user of completing an individual activity as a product of the probability distribution and the engagement value function.

13. The system of claim 12, wherein the NBE engineer is configured to compute the expected value to the user of completing an individual activity by applying a discount factor to the engagement valued function that exponentially diminishes the assigned numerical value to the user in the present of activities completed in future.

14. The system of claim 11, wherein model builder is configured to define the historical user engagement function over a finite period of time.

15. The system of claim 11, wherein model builder is configured to define the historical activity completion function over a finite period of time.

16. The system of claim 11, wherein the plurality of possible activity sequences comprises all possible activity sequences defined for a health program in which the user is enrolled.

17. The system of claim 11, wherein the model builder is configured to:
generate each of the probability distribution and an engagement value function based on the demographic data about the user that is assumed to be static.

18. The system of claim 11, wherein the NBE engine is configured to:
generate a recommended sequence of activities having a length that is constrained by a user activity window and an orchestration window.

19. The system of claim 18, further comprising
a program module configured to transmit, for display on a mobile device operated by the user, a first portion of the recommended sequence of activities defined by the user activity window.

20. The system of claim 19, wherein the program module is configured to transmit, to the mobile device operated by the user, a second portion of the recommended sequence of activities defined by the orchestration window, for transfer into the user activity window as activities are completed by the user.
